(19) United States
(12) Patent Application Publication
(10) Pub. No.: US 2023/0229915 A1
(43) Pub. Date: Jul. 20, 2023

(54) METHOD AND APPARATUS FOR PREDICTING FUTURE STATE AND RELIABILITY BASED ON TIME SERIES DATA

(71) Applicant: ELECTRONICS AND TELECOMMUNICATIONS RESEARCH INSTITUTE, Daejeon (KR)

(72) Inventors: Hwin Dol PARK, Daejeon (KR); Jae Hun CHOI, Daejeon (KR); Young Woong HAN, Daejeon (KR)

(73) Assignee: ELECTRONICS AND TELECOMMUNICATIONS RESEARCH INSTITUTE, Daejeon (KR)

(21) Appl. No.: 18/155,471

(22) Filed: Jan. 17, 2023

(30) Foreign Application Priority Data
Jan. 17, 2022 (KR) 10-2022-0006806

(51) Int. Cl. G06N 3/08

(52) U.S. Cl. CPC G06N 3/08

(57) ABSTRACT

Disclosed herein is a method and apparatus for predicting a future state and reliability based on time series data. In the method and the apparatus, a future state is predicted by preprocessing past state data and executing an algorithm based on the preprocessed past state data to generate a trained model, followed by preprocessing current state data and executing an algorithm based on the created trained model, the preprocessed current state data, and the preprocessed past state data.

### CROSS-REFERENCE TO RELATED APPLICATION

This application claims priority to and the benefit of Korean Patent Application No. 10-2022-0006806 filed on Jan. 17, 2022, the disclosure of which is incorporated herein by reference in its entirety.

### FIELD

Embodiments of the present disclosure relate to a method and apparatus for predicting a future state and reliability, and, more particularly, to a method and apparatus for predicting a future state and reliability based on time series data.

### BACKGROUND

Time series data has features of predicting a future state in consideration of three features, for example, sequentiality, complexity, and time series trend.

First, sequentiality means data sequentially measured over time. For example, a time series length for a patient hospitalized for 10 years is 3,650 days. Since the time series length for an emergency patient hospitalized for 3 days is 3 days, there is discrepancy between these data. In order to solve such a problem, there is a need for an effective analysis method according to the time series length.

Secondly, complexity means data having multiple features instead of only a single feature. For example, numerical data, such as blood pressure, cholesterol level, blood sugar, and the like, and categorical data, such as sex, surgery state, blood type, and the like, are mixed to have multiple features. At a larger time point, data for a high-risk group, a low-risk group, and a normal group are present in different ratios. It is necessary to analyze data with high complexity, such as an acute patient group, a chronic disease group, and an aging patient group, where data of various characteristics are mixed.

Thirdly, time series trend means a trend feature between a measurement time and data. For example, in order to predict a future health state of a patient at a desired point in time based on past and current data of the patient, it is necessary to model a variation rate of the health state of the patient over time. If modeling of a change trend of data over time fails, it is possible to predict only the nearest future state of the patient.

Last, in order for users to utilize results predicted by a system, the prediction results must be reliable. If important decisions can differ depending on the prediction results, such as medical data, a prediction basis and reliability must be provided such that users can trust the prediction results.

However, although a conventional technique provides the prediction basis to allow a user to understand a prediction result of the system, the conventional technique fails to provide certainty about the prediction result.

### SUMMARY

Embodiments of the present disclosure provide a method and apparatus for predicting a future state and reliability, which can predict a future state at a desired point in time with high reliability by modeling time series data features.

The above and other objects and advantages of the present disclosure will become apparent from the following description of embodiments. In addition, it could be easily understood that the objects and advantages of the present disclosure can be implemented by features set forth in the claims and combinations thereof.

In accordance with one aspect of the present disclosure, a method of predicting a future state and reliability based on time series data includes: preprocessing past state data; creating a trained model through execution of an algorithm based on the preprocessed past state data; preprocessing current state data; and predicting a future state through execution of an algorithm based on the created trained model, the preprocessed current state data, and the preprocessed past state.

The step of preprocessing past state data may include: removing outliers from the past data; and calculating a time series length of the past data.

The step of creating a trained model may include: adjusting a training direction of the trained model based on the preprocessed past state data; creating a structure of the trained model based on at least one of the number of training repetition times, a model size, and an algorithm; and creating a trained model by reflecting instability in the created structure of the trained model.

In generation of the trained model by reflecting instability in the created structure of the trained model, the trained model may be created using at least one of percentage instability and instability with reference to a specific critical point.

The step of predicting a future state through execution of an algorithm may include: creating a structure of the trained model; executing an algorithm reflecting a time series feature of the preprocessed past state data in the trained model; processing a prediction point-in-time of the preprocessed past state data; applying a suitable feature to the preprocessed past state data by modeling an environment condition feature; and calculating instability in the course of predicting the future state based on the time series feature of the preprocessed past state data.

The method may further include: applying a suitable feature to the preprocessed past state data by modeling the environment condition feature; creating a complexity distribution of the preprocessed past state data; creating a complexity distribution sampling based on the created complexity distribution; and creating future state data based on the created complexity distribution sampling.

The step of processing a prediction point-in-time of the preprocessed past state data may include: training a function for estimation of a variation rate of input data through deep learning; and calculating a variation estimation function depending on the prediction point-in-time using the function.

The step of calculating instability may include: calculating instability using a weighted sum corresponding to at least one of time series instability, point-in-time instability and distribution complexity instability through deep learning.

The step of predicting a future state may include: calculating a prediction point-in-time by receiving a future point-in-time that a user wants to know; and predicting a future state of the user through execution of an algorithm based on the prediction point-in-time and the trained model.

The step of predicting the future state may include calculating at least one of reliability of the future state, a prediction basis of the future state, and instability.

In accordance with another aspect of the present disclosure, an apparatus for predicting a future state and reliability based on time series data includes: a time series feature preprocessing unit preprocessing at least one of past state data and current state data; a future state prediction model-training unit creating a trained model through execution of an algorithm based on the preprocessed past state data; and a future state prediction unit predicting a future state through execution of an algorithm based on the created trained model, the preprocessed current state data, and the preprocessed past state data.

The time series feature preprocessing unit may include: an outlier-processing device removing outliers from the past data; and a time series length calculator calculating a time series length of the past data.

The future state prediction model-training unit may include: a multiple points-in-time generator adjusting a training direction of the trained model based on the preprocessed past state data; a time series model-training unit creating a structure of the trained model based on at least one of the number of training repetition times, a model size, and an algorithm; and an instability calculator creating a trained model by reflecting instability in the created structure of the trained model.

The instability calculator may generate the trained model using at least one of percentage instability and instability with reference to a specific critical point.

The apparatus for predicting a future state and reliability based on time series data may further include: an algorithm calculator, the algorithm calculator including: a model variable setting device creating a structure of the trained model; a time series feature processing device applying an algorithm reflecting a time series feature of the preprocessed past state data in the trained model; a point-in-time feature processing device processing a prediction point-in-time of the preprocessed past state data; an environment feature processing device applying a suitable feature to the preprocessed past state data by modeling an environment condition feature; and an instability processing device calculating instability in the course of predicting the future state based on the time series feature of the preprocessed past state data.

The environment feature processing device may create a complexity distribution of the preprocessed past state data, a complexity distribution sample based on the created complexity distribution, and future state data based on the created complexity distribution sampling.

The point-in-time feature processing device may trains a function for estimation of a variation rate of input data through deep learning and may calculate a variation estimation function depending on a prediction point-in-time using the trained function for estimation of a variation rate of input data.

The instability processing device may calculate instability using a weighted sum corresponding to at least one of time series instability, point-in-time instability and distribution complexity instability through deep learning.

The future state prediction unit may calculate a prediction point-in-time by receiving a future point-in-time that a user wants to know, and may predict a future state of a user through execution of an algorithm based on the prediction point-in-time and the trained model

In accordance with a further aspect of the present disclosure, an apparatus for predicting a future state and reliability based on time series data includes: a transceiver transmitting and receiving past data and current data to and from an external device; a processor preprocessing at least one of the past state data and the current state data, creating a trained model through execution of an algorithm based on the created trained model, and predicting a future state through execution of an algorithm based on the created trained model, the preprocessed current state data, and the preprocessed past state data; and a memory storing the trained model and the future state.

The method and apparatus for predicting a future state and reliability based on time series data according to embodiments of the present disclosure can predict a future state of a user with high reliability at a desired point in time through modeling of time series data features by preprocessing past state data and current state data and executing corresponding algorithms based on the created trained model, the preprocessed current state data, and the preprocessed past state data, whereby the user can analyze a prediction result of the future state with high reliability.

It should be understood that the present disclosure is not limited to the above effect and other effects and advantages will become apparent from the following description.

### BRIEF DESCRIPTION OF THE DRAWINGS

FIG. 1 is a block diagram of an apparatus for predicting a future state and reliability based on time series data according to one embodiment of the present disclosure.

FIG. 2 is a block diagram of a time series feature preprocessing unit according to the embodiment of the present disclosure.

FIG. 3 is a diagram illustrating an exemplary operation of the time series feature preprocessing unit according to the embodiment of the present disclosure.

FIG. 4 is a block diagram of a future state prediction model-training unit according to one embodiment of the present disclosure.

FIG. 5 is a view illustrating an exemplary operation of a multiple points-in-time generator according to one embodiment of the present disclosure.

FIG. 6 is a view illustrating an exemplary operation of an instability calculator according to one embodiment of the present disclosure.

FIG. 7 is a block diagram of an algorithm calculator according to one embodiment of the present disclosure.

FIG. 8 is a flowchart illustrating a calculation process of the algorithm calculator according to the embodiment of the present disclosure.

FIG. 9 is a view illustrating an exemplary operation of a time series feature processing device according to one embodiment of the present disclosure.

FIG. 10 is a view illustrating an exemplary operation of a point-in-time feature processing device according to one embodiment of the present disclosure.

FIG. 11 is a view illustrating an exemplary operation of an environment feature processing device according to one embodiment of the present disclosure.

FIG. 12 is a view illustrating equations used by the environment feature processing device according to the embodiment of the present disclosure.

FIG. 13 is a view illustrating an exemplary operation of an instability processing device according to one embodiment of the present disclosure.

FIG. 14 is a block diagram of a future state prediction unit according to one embodiment of the present disclosure.

FIG. 15 is a block diagram of a prediction point-in-time calculator according to one embodiment of the present disclosure.

FIG. 16 is a flowchart illustrating a method of predicting a future state and reliability based on time series data according to one embodiment of the present disclosure.

FIG. 17 is a block diagram of an apparatus for predicting a future state and reliability based on time series data according to one embodiment of the present disclosure.

### DETAILED DESCRIPTION

Hereinafter, embodiments of the present disclosure will be described with reference to the accompanying drawings such that the present disclosure can be easily implemented by those skilled in the art. It will be understood that the present disclosure may be embodied in different ways and is not limited to the following embodiments.

Detailed description of components and functions well-known to those skilled in the art is omitted for clarity. In the drawings, portions irrelevant to the description will be omitted for clarity and like components will be denoted by like reference numerals.

Herein, components described as distinct from each other are set forth to clearly describe features thereof and this does not necessarily mean that the components are separated from each other. That is, multiple components may be integrated to form a single hardware or software unit, or a single component may be distributed to form multiple hardware or software units. Therefore, it will be understood that, even if not mentioned separately, such an integrated or distributed embodiment is also included in the scope of the present disclosure.

Herein, components described in various embodiments are not essential and some components may be optional. Therefore, it will be understood that an embodiment composed of a subset of components described in one embodiment is also included in the scope of the present disclosure. In addition, it will be understood that embodiments including other components in addition to components described in various embodiments are also within the scope of the present disclosure.

It will be understood that, although the terms “first”, “second”, and the like may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a “first” element or component discussed below could also be termed a “second” element or component, or vice versa, without departing from the scope of the present disclosure.

It will be understood that, when an element or component is referred to as being “connected to” or “coupled to” another element or component, it may be directly connected or coupled to the other element or component, or intervening elements or components may be present. In contrast, when an element or component is referred to as being “directly connected to” or “directly coupled to” another element or component, there may be no intervening elements or components therebetween.

Further, description of each drawing may be applied to different drawings, so long as one drawing illustrating an embodiment of the present disclosure does not correspond to another drawing and an alternative embodiment.

Hereinafter, embodiments of the present disclosure will be described with reference to the accompanying drawings.

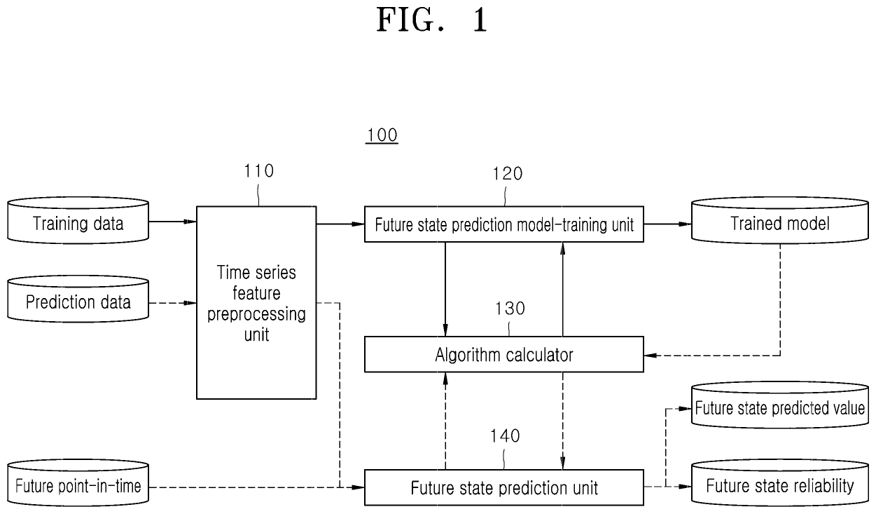

FIG. 1 is a block diagram of an apparatus for predicting a future state and reliability based on time series data according to one embodiment of the present disclosure.

Referring to FIG. 1, a future state/reliability prediction apparatus 100 includes a time series feature preprocessing unit 110, a future state prediction model-training unit 120, an algorithm calculator 130, and a future state prediction unit 140.

A process according to the present disclosure may be divided into a training process and a prediction process.

First, the training process refers a process of creating a future state prediction model. A trained model is created through sequential operation of the time series feature preprocessing unit 110, the future state prediction model-training unit 120 and the algorithm calculator 130 based on past state recording data having time series features.

The prediction process provides a future state prediction value and future state reliability through sequential operation of the time series feature preprocessing unit 110, the time series feature preprocessing unit 110, the future state prediction unit 140, and the algorithm calculator 130 based on past state data, current state data, and a desired point in time that a user wants to know.

Next, a function of each unit will be described below in detail.

The time series feature preprocessing unit 110 preprocesses past state data and/or current state data.

The future state prediction model-training unit 120 creates a trained model through execution of an algorithm based on the preprocessed past state data.

The future state prediction unit 140 predicts a future state by executing an algorithm based on the created trained model, the preprocessed current state data, and the preprocessed past state data.

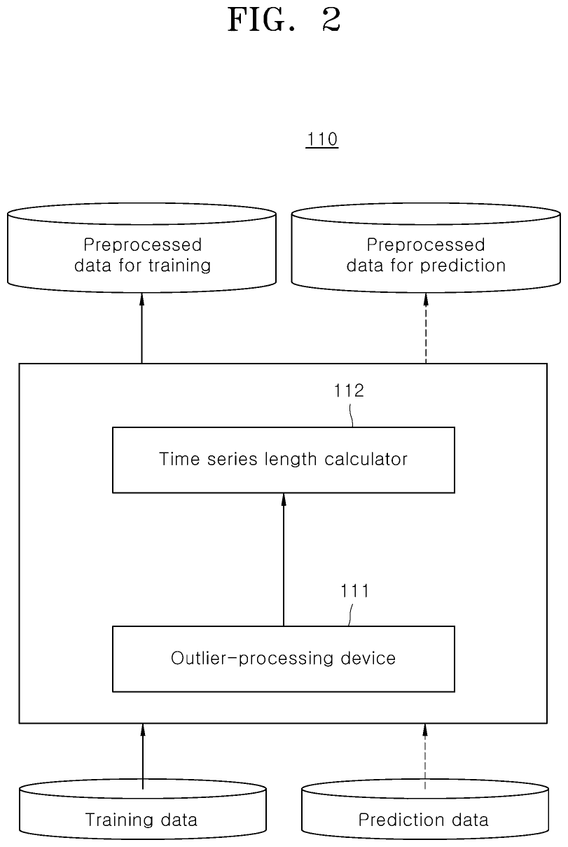

FIG. 2 is a block diagram of the time series feature preprocessing unit according to the embodiment of the present disclosure.

Referring to FIG. 2, the time series feature preprocessing unit 110 performs both the training process and the prediction process, and includes an outlier-processing device 111 and a time series length calculator 112.

The outlier-processing device 111 removes outliers from past data.

The time series length calculator 112 calculates a time series length of the past data.

The outlier-processing device 111 performs a process of removing and replacing outliers, such as missing values, specific values, and abnormal values, which are present in data. The replacing process means a process of processing data using various values, such as 0, a previous value, and an average value.

The time series length calculator 112 calculates the total time series length of each set of sample data, for example, data of one patient. The time series length is information to reflect characteristics of data according to time series length in calculation by the algorithm calculator 130. Detail of this process will be described in description of the algorithm calculator.

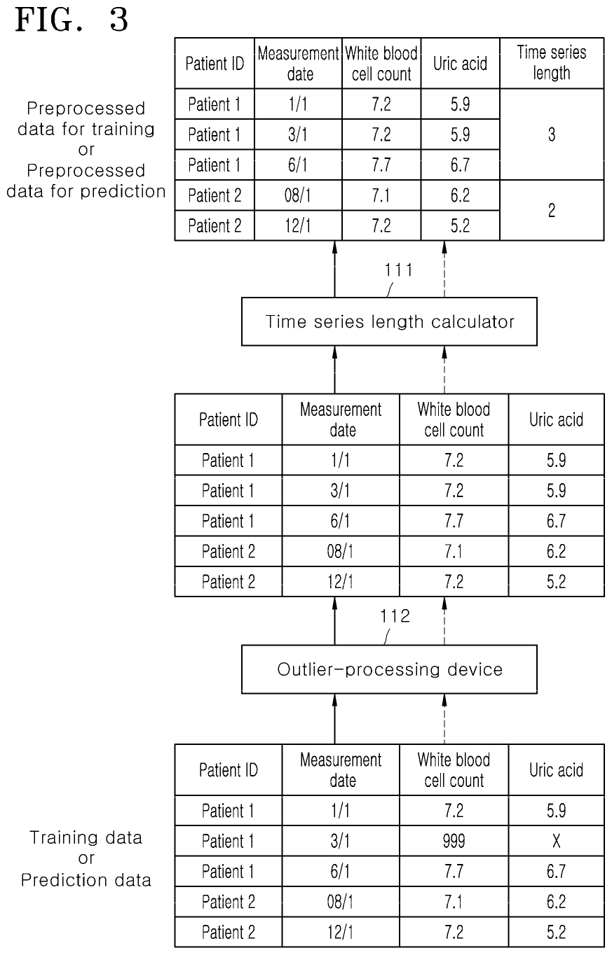

FIG. 3 is a diagram illustrating an exemplary operation of the time series feature preprocessing unit according to the embodiment of the present disclosure.

Referring to FIG. 3, the outlier-processing device 111 receives training data or prediction data, and removes and replaces outliers, such as missing values, specific values, and abnormal values, which are present in the data.

The time series length calculator 112 calculates the total time series length of data of one patient. For example, the time series length of data of patient 1 is 3. The time series length of data of patient 2 is 2.

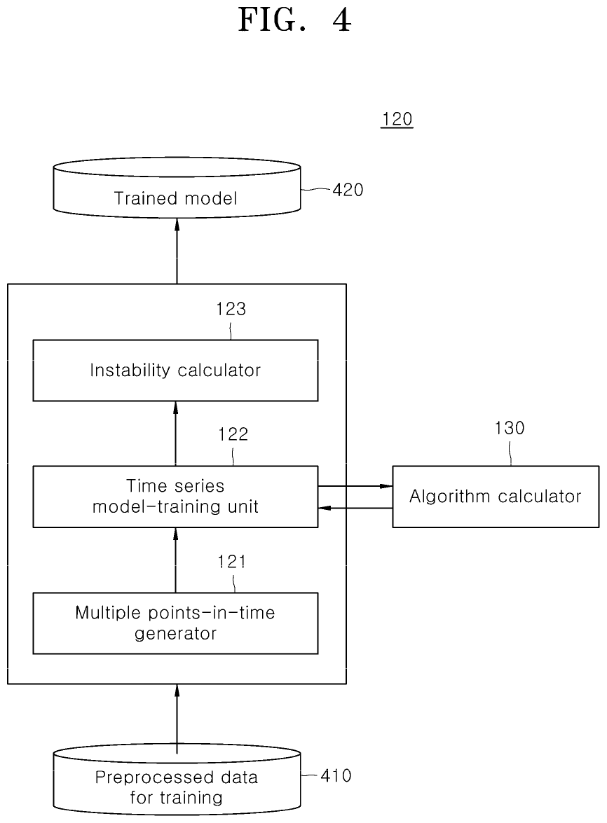

FIG. 4 is a block diagram of the future state prediction model-training unit according to the embodiment of the present disclosure.

Referring to FIG. 4, the future state prediction model-training unit 120 creates a trained model 420 by receiving preprocessed data 410 for model training. The future state prediction model-training unit 120 is composed of a multiple points-in-time generator 121, a time series model-training unit 122, and an instability calculator 123.

The multiple points-in-time generator 121 adjusts a training direction of the trained model based on the preprocessed past state data.

The time series model-training unit 122 creates a structure of the trained model based on at least one of the number of training repetition times, a model size, and an algorithm.

The time series model-training unit 122 sends an output to the algorithm calculator 130.

The instability calculator 123 creates a trained model by reflecting instability in the created structure of the trained model.

The instability calculator 123 creates the trained model by reflecting at least one of percentage instability and instability with reference to a specific critical point.

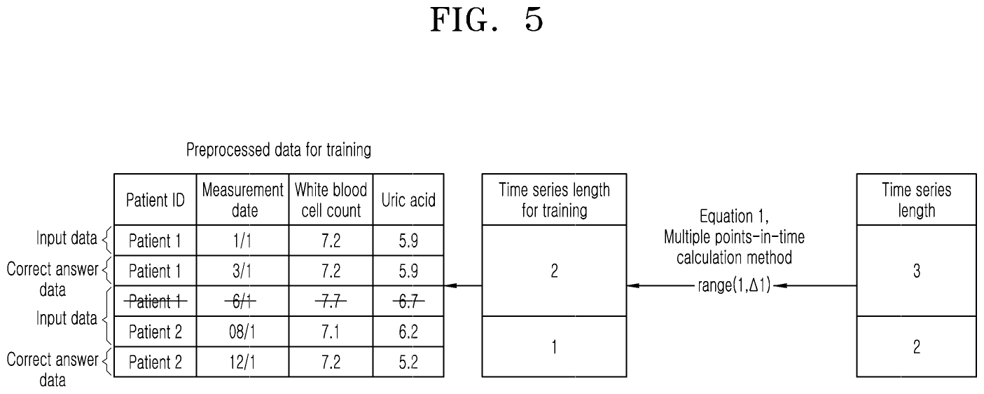

FIG. 5 is a view illustrating an exemplary operation of the multiple points-in-time generator according to the embodiment of the present disclosure.

Referring to FIG. 5, the multiple points-in-time generator 121 serves to classify input data and correct data of each set of sample data. In the course of training a model, the past data is input to adjust the training direction of the model through calculation of an error between a prediction value and an actual value in prediction of a future state. Since there is no correct answer in actual prediction of the future state, the error is calculated on the assumption that a specific point in time in the past data is a future point-in-time.

A training point-in-time of each set of sample data is determined through a multiple points-in-time calculation method based on the time series length calculated by the time series feature preprocessing unit 110. As in Equation 1, the multiple points-in-time calculation method derives a random integer value from 1 to less than the time series length.

For example, Patient 1 visits a hospital a total of 3 times, providing data having 3 records. In general, the last visit point-in-time is used as correct answer data and two previous data are used as input data for model training.

In such a training method, it is difficult to learn various prediction points in time due to limited data. If some patients visited a hospital about 5 times in the past, the model will become a model that predicts data at the 5^{th} visit point-in-time based on input data of the 4 previous visits. In addition, if the fifth visit point-in-time of most patients is 1 year later, it is possible to predict a point in time in the future by training data features only at the point in time 1 year later. In order for the model to predict various points in time, it is necessary to learn data features at various points in time. To solve this problem, it is possible to increase diversity of data and applicability of the model by randomly configuring the training point-in-time in limited data.

The time series model-training unit 122 creates a model structure according to conditions set by a user or a manager. The time series model-training unit 122 creates the model through the algorithm calculator 130 based on conditions, such as the number of training repetition times, a model size, and an optimization algorithm, and performs training through the preprocessed data for training. The model trained by the time series model-training unit 122 will be referred to as an algorithm model to describe the instability calculator.

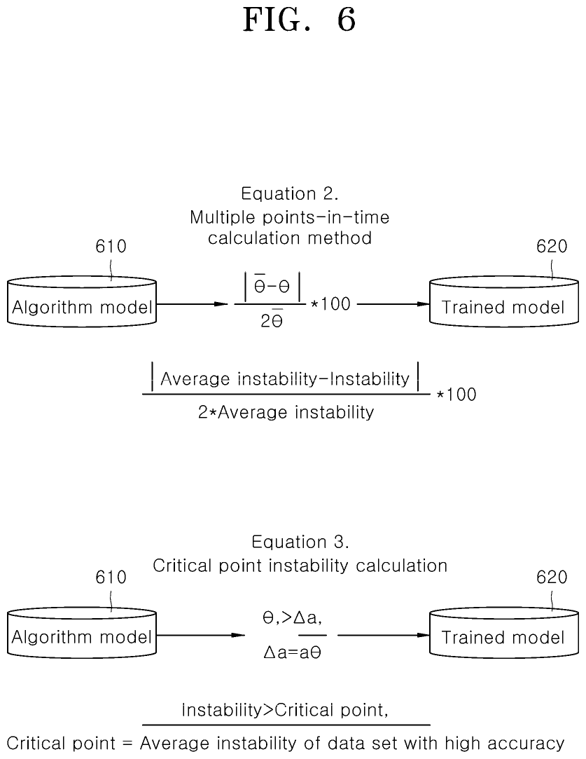

FIG. 6 is a view illustrating calculation methods of the instability calculator according to the embodiment of the present disclosure.

Referring to FIG. 6, the instability calculator 123 creates a finally trained model 620 by reflecting instability in the algorithm model trained by the time series model-training unit 122. Here, the instability refers to an index for calculation of reliability of the model and is created by the instability processing device in the algorithm calculator 130.

For calculation of the instability, an instability reference is calculated by the instability calculator to estimate whether a current prediction value is high or low.

Herein, reliability is determined by calculating the instability with reference to examples.

First, a percentage instability calculation method will be described.

In this method, a distance from current instability to average instability of all of learned data is calculated as a percentage with respect to the average instability, as in Equation 2. Since the current instability is obtained as a percentage in the range of 1% to 100%, users can easily understand prediction reliability.

Next, a critical point instability calculation method will be described.

In this method, reliability of the prediction result is determined with reference to a specific critical point, as in Equation 3. In this method, average instability is obtained by forming a data set with high accuracy. The data set with high accuracy is formed by calculating instability levels through a model of data having a low error rate between the prediction value and the correct answer, followed by averaging the instability levels.

According to the present disclosure, users can intuitively understand prediction reliability using both methods as described above.

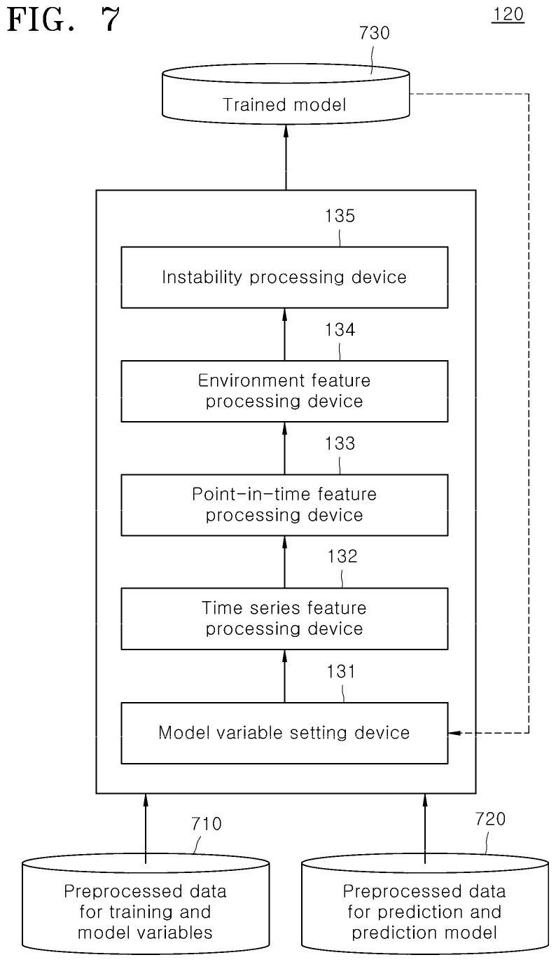

FIG. 7 is a block diagram of the algorithm calculator according to the embodiment of the present disclosure.

Referring to FIG. 7, the algorithm calculator 130 performs modeling of time series features and instability. Here, the time series features refer to sequentiality, complexity, and time series trend.

The algorithm calculator 130 receives preprocessed data for training, model variables 710, preprocessed data for prediction, and a prediction model 720, and outputs a trained model 730.

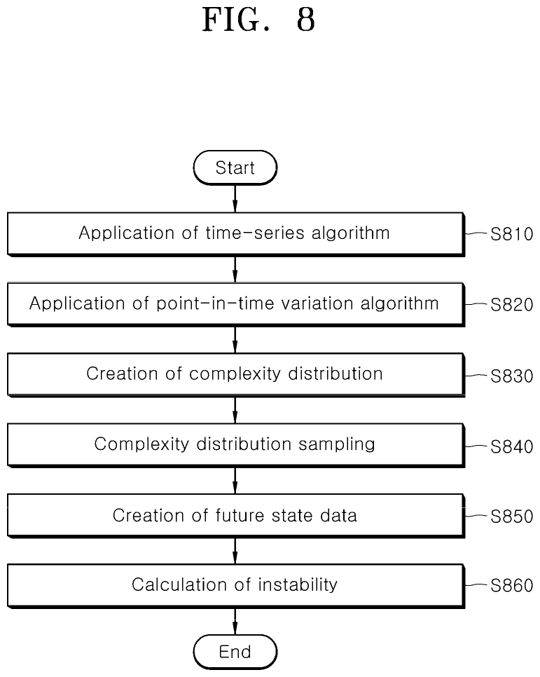

The algorithm calculator 130 is composed of a model variable setting device 131, a time series feature processing device 132, a point-in-time feature processing device 133, an environment feature processing device 134, and an instability processing device 135. FIG. 8 is a flowchart of an algorithm. Details of the flowchart will be described in description of individual components.

The model variable setting device 131 creates a structure of the trained model.

The time series feature processing device 132 adopts an algorithm which reflects the time series features of the preprocessed past state data in the trained model.

The point-in-time feature processing device 133 processes the prediction point-in-time of the preprocessed past state data.

The point-in-time feature processing device 133 learns a function for estimation of a variation rate of input data through deep learning and calculates a variation estimation function using the function for estimation of the variation rate of input data.

The environment feature processing device 134 applies a suitable feature to the preprocessed past state data by modeling an environment condition feature.

The environment feature processing device 134 creates a complexity distribution of the preprocessed past state data, a complexity distribution sample based on the created complexity distribution, and future state data based on the created complexity distribution sampling.

The instability processing device 135 calculates instability in the course of predicting a future state based on the time series feature of the preprocessed past state data.

The instability processing device 135 calculates instability using a weighted sum corresponding to at least one of time series instability, point-in-time instability and distribution complexity instability through deep learning.

FIG. 8 is a flowchart illustrating a calculation process of the algorithm calculator according to the embodiment of the present disclosure.

Referring to FIG. 8, the model variable setting device 131 creates the overall structure of the model or retrieves the trained model. For training, the model variable setting device 131 creates a model structure by receiving a model size, a function to be used, and a training rate from a user or default settings. For prediction, a model selected by a user is retrieved from trained models.

First, a time-series algorithm is applied (S810).

A point-in-time variation algorithm is applied (S820).

A complexity distribution is created (S830).

A complexity distribution sample is created (S840).

Future state data is created (S850).

Instability is calculated (S860).

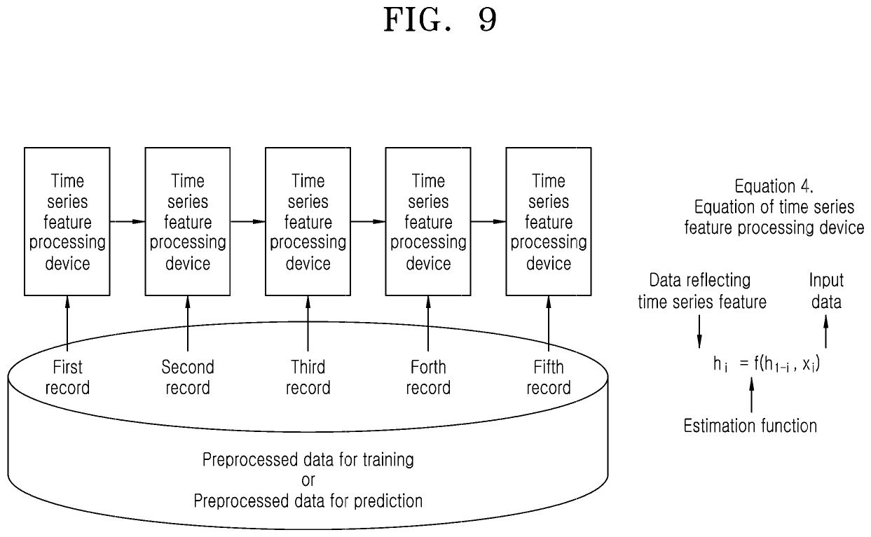

FIG. 9 is a view illustrating an exemplary operation of the time series feature processing device according to the embodiment of the present disclosure.

Referring to FIG. 9, the time series feature processing device 132 applies an algorithm that can reflect a time series feature in the model.

In Equation 4, xᵢ denotes input data at a current point in time. hᵢ denotes input data reflecting the time series feature. hᵢ₋₁ denotes input data at a previous point in time, with the time series feature reflected therein.

The algorithm calculates an estimation function, which can find a sequence pattern between the input data at the current point in time and the input data at the previous point in time, based on deep learning, and creates input data that reflects the time series feature, as in Equation 4.

The time-series algorithm may be an algorithm, such as an auto-regressive integrated moving average (ARIMA) or recurrent neural network (RNN) deep learning-based algorithm. FIG. 8 is shown to sequentially reflect the time series feature based on RNN. The data reflecting the time series feature is delivered to the point-in-time feature processing device 133.

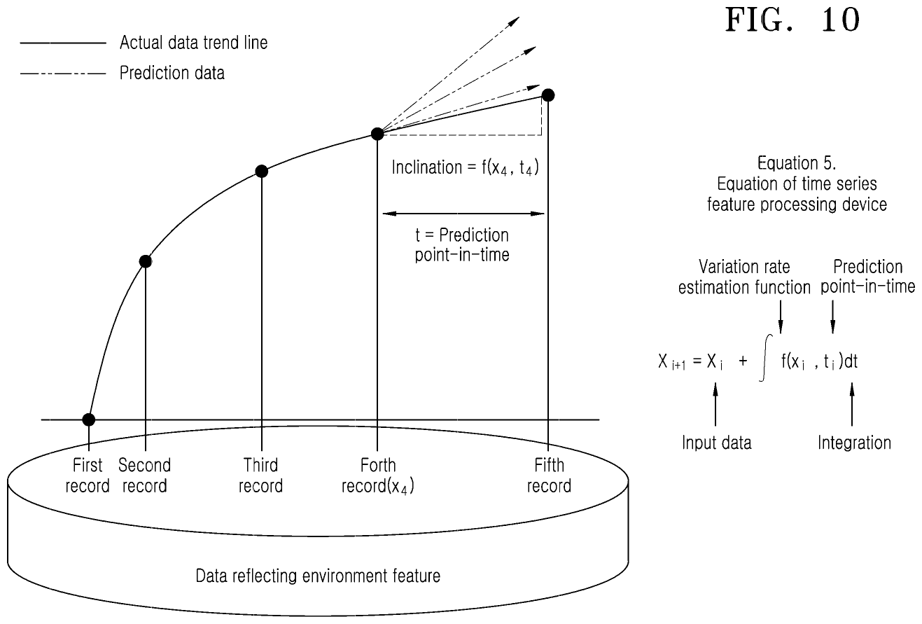

FIG. 10 is a view illustrating an exemplary operation of the point-in-time feature processing device according to the embodiment of the present disclosure.

Referring to FIG. 10, the point-in-time feature processing device 133 processes a prediction point-in-time of the preprocessed past state data. The point-in-time feature processing device 133 applies time series transitivity according to the prediction point-in-time.

In Equation 5, xᵢ₊₁ denotes predicted future data and xᵢ denotes input data.

For application of transitivity, the point-in-time feature processing device learns an f-function that estimates the variation rate of the input data according to the prediction point-in-time through deep learning, as shown in Equation 5. The f-function is a function estimating a differential coefficient for calculation of the variation rate of the input data that continuously changes according to the prediction point-in-time, like the structure of a differential equation. When the variation rate of the input data obtained through the f-function is added to the input data, the future data of the prediction point-in-time can be predicted. Deep learning trains a model by adjusting the coefficient of the f-function so as to minimize the difference between predicted future data and actual data.

A function for obtaining the variation change according to the prediction point-in-time may be calculated using the trained function.

Here, the prediction point-in-time means a time difference between the last point-in-time of input data and a point-in-time to be predicted.

Through the point-in-time feature processing device 133, it is possible to predict a future state at a user's desired point in time. The point-in-time feature processing device 133 sends data, which reflects a prediction point-in-time, to the environment feature processing device 134.

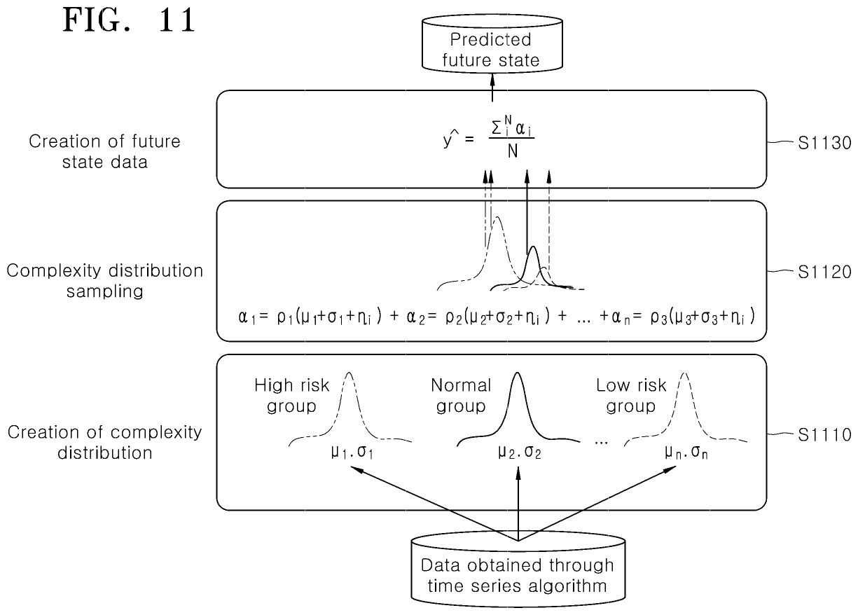

FIG. 11 is a view illustrating an exemplary operation of the environment feature processing device according to the embodiment of the present disclosure.

Referring to FIG. 11, the environment feature processing device 134 applies suitable features according to input data by modeling various environment condition features in the data set. The environment feature processing device 134 may predict an accurate future state of a patient.

The environment feature processing device 130 creates a complexity distribution S1110.

The environment feature processing device creates a complexity distribution sample (S1120).

The environment feature processing device creates a future state (S1130).

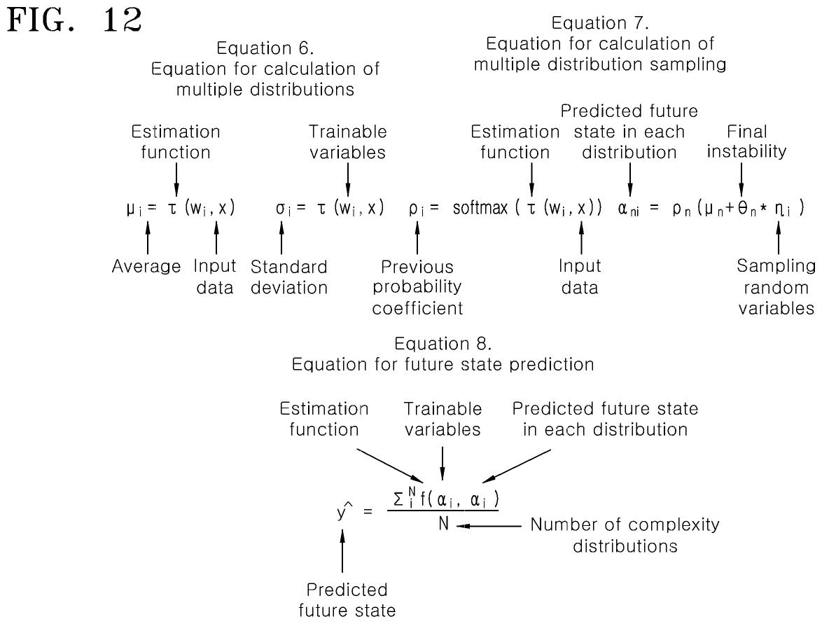

FIG. 12 is a view illustrating equations used by the environment feature processing device according to the embodiment of the present disclosure.

In FIG. 12, Equation 6 is an equation for calculation of multiple distributions. Equation 7 is an equation for calculation of multiple-distribution sampling. Equation 8 is an equation for future state prediction.

Multiple patient groups may be present. For example, the patient groups include high-risk patients who have visited a hospital for acute disease, low-risk patients who have visited the hospital for small wounds, and normal patients who have visited the hospital for health checkups.

A data set for high-risk patients may include various patient groups, such as patients with chronic diseases and patients with acute diseases. When past health state data of a specific patient is input in order to predict a future health state of the specific patient, the future state should be predicted in consideration of suitable patient group characteristics of a current condition of the patient.

To this end, the environment feature processing device 134 creates a complexity distribution. As shown in Equation 6, multiple means and standard deviations may be estimated from the complexity distribution.

An estimation function is a deep learning-based nonlinear function. A function for estimation of the means and the standard deviations is created through deep learning.

A process of estimating the function is a process of transforming a predicted distribution into an assumed distribution while reducing an error of a distance function of the predicted distribution, that is, the mean and the standard distribution, with respect to the assumed distribution, for example, a normal distribution, based on an assumption that the function is one of various distributions, such as normal distribution, gamma distribution, and the like. Here, the distance function may be KL divergence.

The future state is predicted by sampling among the created multiple distributions. Here, a distribution suitable for input data is retrieved through the function for estimation of a private probability coefficient.

A function for estimation of a previous probability coefficient with input data and learnable variables is retrieved through deep learning, as in Equation 7. Here, the resulting value may be obtained as a probability through an activation function capable of estimating a probability that the resulting value belongs to each class, such as SoftMax. The probability that current data belongs to a corresponding distribution is calculated by multiplying each distribution sample value by the corresponding probability.

Here, Distribution sampling value=Mean+Final instability×Sampling noise.

Finally predicted future state data may be obtained by averaging predicted future state data for each distribution calculated in this way or by applying a weighted average, as in Equation 8.

The basis of prediction may also be estimated through such an algorithm. It is possible to estimate the basis for prediction through a feature data set by inversely tracing a distribution to which the corresponding data set refers most. For example, the most referenced distribution obtained using a high-risk patient data set may be a distribution that estimates the high-risk group. The instability processing device 135 calculates final instability.

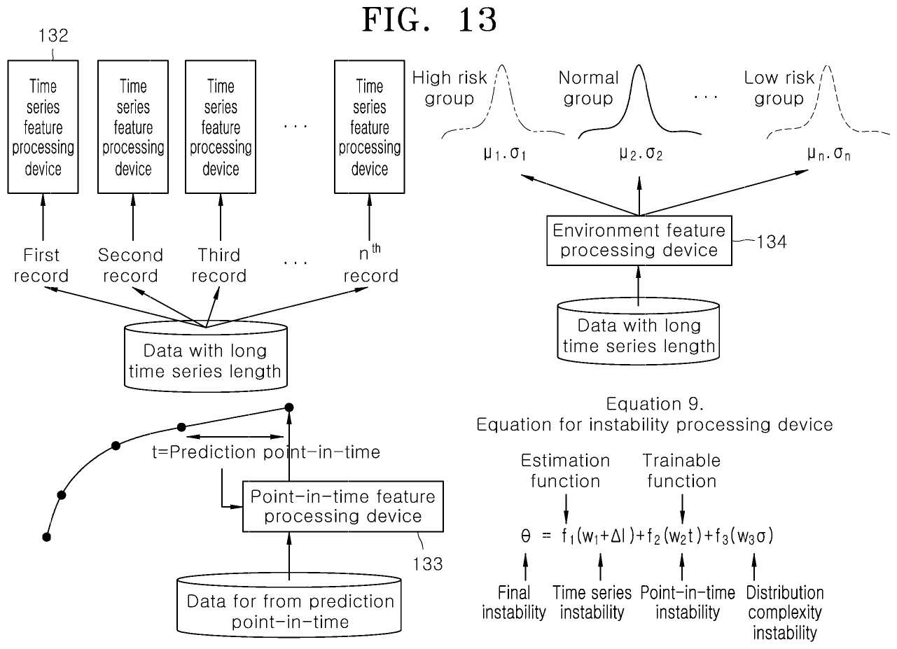

FIG. 13 is a view illustrating an exemplary operation of the instability processing device according to the embodiment of the present disclosure.

Referring to FIG. 13, the instability processing device 135 provides reliability of a predicted result value by calculating instability due to estimation in the course of predicting a future state in consideration of the time series feature.

In a method of calculating final instability as in Equation 9, the final instability may be calculated by adding all weighted values estimated corresponding to importance of time series instability, point-in-time instability, and distribution complexity instability through deep learning.

In Equation 7, the final instability is used when obtaining the final predicted future state data to induce estimation functions to be learned. The time series instability is a function for calculation of instability due to too long or too short time series lengths.

The point-in-time instability is a function that reflects features that can appear when a prediction point-in-time is too far in the future. In addition, the distribution complexity instability is a function that reflects instability making it difficult to achieve proper modeling of a characteristic environment due to the presence of too many characteristic environments relative to the size of the data set.

In the training process, the algorithm model trained through the above process is delivered to the future state prediction model-training unit 120. In the prediction process, a finally predicted future state, a prediction basis, and final instability are delivered to the future state prediction unit 140.

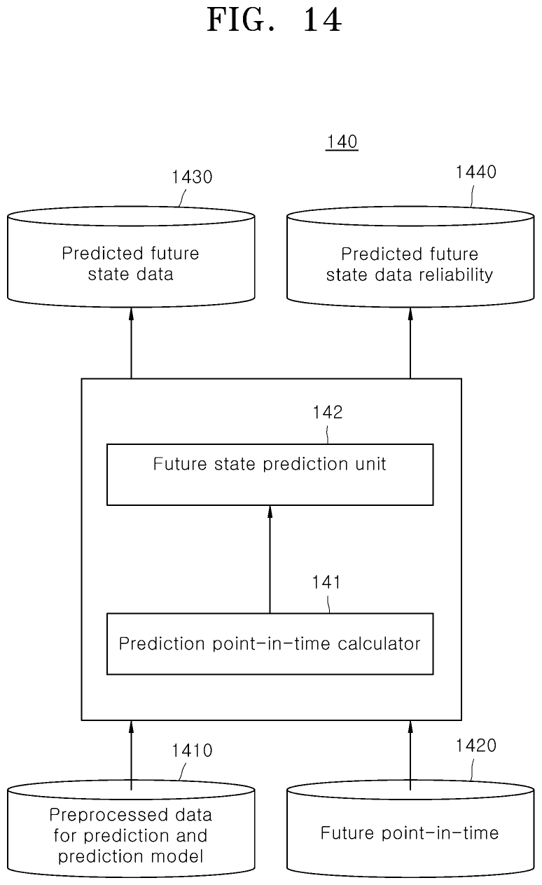

FIG. 14 is a block diagram of the future state prediction unit according to the embodiment of the present disclosure.

Referring to FIG. 14, the future state prediction unit 140 includes a prediction point-in-time calculator 141 and a future state prediction unit 142.

The future state prediction unit 140 receives prediction-preprocessed data, a prediction model 1410 and a future point-in-time 1420, and outputs predicted future state data 1430 and predicted future state data reliability 1440.

The future state prediction unit 140 predicts a future state at a point in time that a user wants to know.

The future state prediction unit 140 calculates a prediction point-in-time by receiving a point in time that a user wants to know, and predicts a user's future state through execution of an algorithm based on the prediction point-in-time and the training model.

The prediction point-in-time calculator 141 calculates the prediction point-in-time by receiving the future point-in-time 1420 that a user wants to know. The calculated prediction point-in-time and prediction data are delivered to the future state prediction unit 142.

The future state prediction unit 142 sends a user location or a present location of the trained model to the algorithm calculator, and receives the predicted future state, the prediction basis, and final instability. The final instability is calculated based on the predicted future state reliability 1440 by converting the index learned by the instability calculator into percentages in comprehensive consideration of whether or not the critical value has been passed together with the prediction basis.

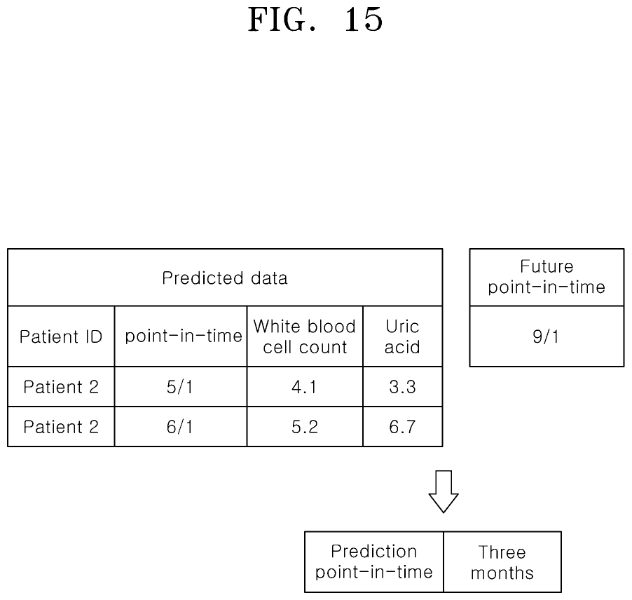

FIG. 15 is a block diagram of the prediction point-in-time calculator according to the embodiment of the present disclosure.

Referring to FIG. 15, for example, for patient 2 who has measurement data including a white blood cell count of 4.1 and a uric acid level of 3.3 as of May 1 and measurement data including a white blood cell count of 5.2 and a uric acid level of 6.7 as of June 1, if the future time point is September 1, the prediction point-in-time is 3 months.

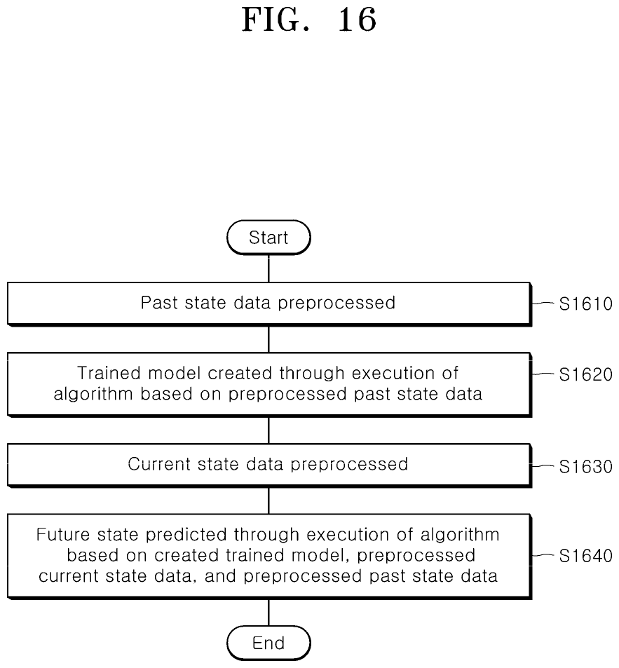

FIG. 16 is a flowchart illustrating a method of predicting a future state and reliability based on time series data according to one embodiment of the present disclosure. The method according to this embodiment is carried out by the apparatus for predicting a future state and reliability.

Referring to FIG. 16, past state data is preprocessed (S1610).

A trained model is created through execution of an algorithm based on the preprocessed past state data (S1620).

Current state data is preprocessed (S1630).

A future state is predicted through execution of an algorithm based on the created trained model, the preprocessed current state data, and the preprocessed past state data (S1640).

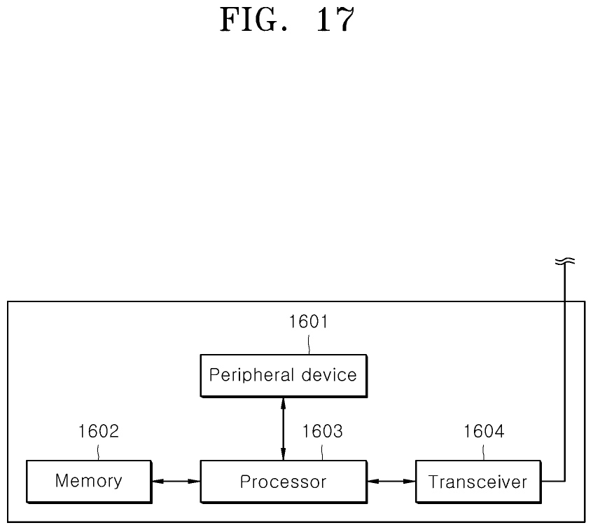

FIG. 17 is a block diagram of an apparatus for predicting a future state and reliability based on time series data according to one embodiment of the present disclosure.

An embodiment of the apparatus 100 for predicting a future state and reliability based on time series data shown in FIG. 1 may be a device 1600. Referring to FIG. 17, the device 1600 may include a memory 1602, a processor 1603, a transceiver 1604, and a peripheral device 1601. In addition, by way of example, the device 1600 may further include other configurations and is not limited thereto.

More specifically, the device 1600 shown in FIG. 17 may be an exemplary hardware/software architecture, such as an apparatus for predicting a future state and reliability, a state prediction device, and the like. Here, by way of example, the memory 1602 may be a non-movable memory or a movable memory. In addition, by way of example, the peripheral device 1601 may include a display, GPS, or other peripheral devices, and is not limited thereto.

Further, by way of example, the device 1600 may include a communication circuit, such as the transceiver 1604, and may perform communication with external devices therethrough.

Further, by way of example, the processor 1603 may include at least one selected from among a universal processor, a digital signal processor (DSP), a DSP core, a controller, a microcontroller, application specific integrated circuits (ASICs), field programmable gate array (FPGA) circuits, any other types of integrated circuits (ICs), and a microprocessor associated with a finite state machine. That is, the processor 1603 may be a hardware/software configuration that performs a control role for controlling the device 1600 described above.

The processor 1603 may execute computer-executable instructions stored in the memory 1602 to perform various essential functions of the apparatus for predicting a future state and reliability based on time series data. For example, the processor 1603 may control at least one of signal coding, data processing, power control, input/output processing, and communication operations. Further, the processor 1603 may control a physical layer, a MAC layer, and an application layer. Further, by way of example, the processor 1603 may perform authentication and security procedures in an access layer and/or an application layer and is not limited thereto.

By way of example, the processor 1603 may communicate with other devices through the transceiver 1604. For example, the processor 1603 may control a 3D model replication device to communicate with other devices through a network by execution of computer-executable instructions. That is, communication performed in the present disclosure may be controlled. For example, the transceiver 1604 may transmit an RF signal through an antenna and may transmit the signal based on various communication networks.

Further, by way of example, as antenna technology, MIMO technology, beamforming, and the like may be applied, without being limited thereto. In addition, signals transmitted and received through the transceiver 1604 may be modulated and demodulated to be controlled by the processor 1603, without being limited thereto.

Various embodiments of the present disclosure are intended to describe representative aspects of the present disclosure rather than listing all possible combinations, and matters described in various embodiments may be applied independently or in combination of two or more.

Further, various embodiments of the present disclosure may be implemented by hardware, firmware, software, or combinations thereof. Hardware may be implemented by one or more application specific integrated circuits (ASICs), digital signal processors (DSPs), digital signal processing devices (DSPDs), programmable logic devices (PLDs), field programmable gate arrays (FPGAs), general processors, controllers, microcontrollers, microprocessors, and the like. For example, it is obvious that various embodiments of the present disclosure may be implemented in the form of a program stored in a non-transitory computer readable medium that can be used at a terminal or at an edge, or in the form of a program stored in a non-transitory computer readable medium that can be used at the edge or in the cloud. Alternatively, various embodiments of the present disclosure may be implemented through a combination of various hardware and software.

The scope of the present disclosure includes software or machine-executable instructions (for example, operating systems, applications, firmware, programs, and the like) that allow operations according to methods of various embodiments to be executed on a device or a computer, and a non-transitory computer-readable medium which stores such software or instructions to be executed on a device or a computer.

Although some embodiments have been described herein, it should be understood that these embodiments are provided for illustration only and are not to be construed in any way as limiting the present disclosure, and that various modifications, changes, alterations, and equivalent embodiments can be made by those skilled in the art without departing from the spirit and scope of the invention. Therefore, the scope of the present disclosure should be defined by the appended claims.

1. A method of predicting a future state and reliability based on time series data, comprising:
preprocessing past state data;
creating a trained model through execution of an algorithm based on the preprocessed past state data;
preprocessing current state data; and
predicting a future state through execution of an algorithm based on the created trained model, the preprocessed current state data, and the preprocessed past state data.

2. The method according to claim 1, wherein the step of preprocessing past state data comprises: removing outliers from the past data; and calculating a time series length of the past data.

3. The method according to claim 1, wherein the step of creating a trained model comprises:
adjusting a training direction of the trained model based on the preprocessed past state data;
creating a structure of the trained model based on at least one of the number of training repetition times, a model size, and an algorithm; and
creating a trained model by reflecting instability in the created structure of the trained model.

4. The method according to claim 3, wherein, in generation of the trained model by reflecting instability in the created structure of the trained model, the trained model is created using at least one of percentage instability and instability with reference to a specific critical point.

5. The method according to claim 1, wherein the step of predicting a future state through execution of an algorithm comprises:
creating a structure of the trained model;
executing an algorithm reflecting a time series feature of the preprocessed past state data in the trained model;
processing a prediction point-in-time of the preprocessed past state data;
applying a suitable feature to the preprocessed past state data by modeling an environment condition feature; and
calculating instability in the course of predicting the future state based on the time series feature of the preprocessed past state data.

6. The method according to claim 5, further comprising:
applying the suitable feature to the preprocessed past state data by modeling the environment condition feature;
creating a complexity distribution of the preprocessed past state data;
creating a complexity distribution sample based on the created complexity distribution; and
creating future state data based on the created complexity distribution sampling.

7. The method according to claim 5, wherein the step of processing a prediction point-in-time of the preprocessed past state data comprises:
training a function for estimation of a variation rate of input data through deep learning; and
calculating a variation estimation function depending on the prediction point-in-time using the function.

8. The method according to claim 5, wherein the step of calculating instability comprises:
calculating instability using a weighted sum corresponding to at least one of time series instability, point-in-time instability and distribution complexity instability through deep learning.

9. The method according to claim 1, wherein the step of predicting a future state comprises:
calculating a prediction point-in-time by receiving a future point-in-time that a user wants to know; and
predicting a future state of the user through execution of an algorithm based on the prediction point-in-time and the trained model.

10. The method according to claim 9, wherein the step of predicting the future state comprises: calculating at least one of reliability of the future state, a prediction basis of the future state, and instability.

11. An apparatus for predicting a future state and reliability based on time series data, comprising:
a time series feature preprocessing unit preprocessing at least one of past state data and current state data;
a future state prediction model-training unit creating a trained model through execution of an algorithm based on the preprocessed past state data; and
a future state prediction unit predicting a future state through execution of an algorithm based on the created trained model, the preprocessed current state data, and the preprocessed past state data.

12. The apparatus according to claim 11, wherein the time series feature preprocessing unit comprises:
an outlier-processing device removing outliers from the past data; and
a time series length calculator calculating a time series length of the past data.

13. The apparatus according to claim 11, wherein the future state prediction model-training unit comprises:
a multiple points-in-time generator adjusting a training direction of the trained model based on the preprocessed past state data;
a time series model-training unit creating a structure of the trained model based on at least one of the number of training repetition times, a model size, and an algorithm; and
an instability calculator creating a trained model by reflecting instability in the created structure of the trained model.

14. The apparatus according to claim 13, wherein the instability calculator creates the trained model using at least one of percentage instability and instability with reference to a specific critical point.

15. The apparatus according to claim 11, further comprising: an algorithm calculator, the algorithm calculator comprising:
a model variable setting device creating a structure of the trained model;
a time series feature processing device applying an algorithm reflecting a time series feature of the preprocessed past state data in the trained model;
a point-in-time feature processing device processing a prediction point-in-time of the preprocessed past state data;
an environment feature processing device applying a suitable feature to the preprocessed past state data by modeling an environment condition feature; and
an instability processing device calculating instability in the course of predicting the future state based on the time series feature of the preprocessed past state data.

16. The apparatus according to claim 15, wherein the environment feature processing device creates a complexity distribution of the preprocessed past state data, a complexity distribution sample based on the created complexity distribution, and future state data based on the created complexity distribution sampling.

17. The apparatus according to claim 15, wherein the point-in-time feature processing device trains a function for estimation of a variation rate of input data through deep learning and calculates a variation estimation function depending on a prediction point-in-time using the trained function for estimation of a variation rate of input data.

18. The apparatus according to claim 15, wherein the instability processing device calculates instability using a weighted sum corresponding to at least one of time series instability, point-in-time instability and distribution complexity instability through deep learning.

19. The apparatus according to claim 11, wherein the future state prediction unit calculates a prediction point-in-time by receiving a future point-in-time that a user wants to know, and predicts a future state of a user through execution of an algorithm based on the prediction point-in-time and the trained model.

20. An apparatus for predicting a future state and reliability based on time series data, comprising:
a transceiver transmitting and receiving past data and current data to and from an external device;
a processor preprocessing at least one of the past state data and the current state data, creating a trained model through execution of an algorithm based on the created trained model, and predicting a future state through execution of an algorithm based on the created trained model, the preprocessed current state data, and the preprocessed past state data; and
a memory storing the trained model and the future state.
